(19) United States
(12) Patent Application Publication
(10) Pub. No.: US 2024/0307041 A1
(43) Pub. Date: Sep. 19, 2024

(54) SAMPLE COLLECTION DEVICES AND KITS, COMPONENTS, AND USES THEREOF

(71) Applicant: Sensible Diagnostics Inc., San Dimas, CA (US)

(72) Inventors: Ernest TEMPLIN, Los Angeles, CA (US); Vladimir I. SLEPNEV, Brunswick, OH (US); Daniel BRAUN, Bend, OR (US); Jaime MARTINEZ, Carlsbad, CA (US)

(21) Appl. No.: 18/282,777

(22) PCT Filed: Mar. 18, 2022
(86) PCT No.: PCT/US2022/020883

§ 371 Date: Sep. 18, 2023

Related U.S. Application Data
(60) Provisional application No. 63/163,631, filed on Mar. 19, 2021.

(51) Int. Cl. A61B 10/00; A61B 10/02; B01L 3/00

(52) U.S. Cl. CPC A61B 10/0096; A61B 10/02; B01L 3/5029; B01L 2200/0615; B01L 2200/0689; B01L 2300/042; B01L 2300/044; B01L 2300/0832

(57) ABSTRACT

The present disclosure, in some aspects, provides sample collection devices useful for collection of a sample such as by a human. In other aspects, the present disclosure is directed to kits comprising the sample collection devices described herein, and components thereof, and methods of using the sample collection devices described herein.

### CROSS REFERENCE TO RELATED APPLICATION

This application claims priority from U.S. Provisional Application No. 63/163,631 filed Mar. 19, 2021, entitled “SAMPLE COLLECTION DEVICES AND KITS, COMPONENTS, AND USES THEREOF,” the contents of which are incorporated herein by reference in their entireties for all purposes.

### TECHNICAL FIELD

The present disclosure, in some aspects, is directed to sample collection devices useful for the collection of a sample, e.g., a sample containing a biological material. In other aspects, the present disclosure is directed to kits comprising the sample collection devices described herein, and components thereof, and methods of using the sample collection devices described herein.

### BACKGROUND

Rapid and efficient analysis of a biological sample is increasingly becoming an essential aspect of modern medicine and responses to infectious disease identification, diagnosis, and containment. Instrumental in this process is the manner in which the biological samples are collected and processed. Conventional techniques using open tubes and a collection tool may leave a consumer/patient susceptible to hazardous reagents in the open tube useful for treating the biological sample and also require additional or low through-put downstream processing steps, such as to remove and/or control the collection tool so that the biological sample can be obtained from the open tube.

All references cited herein, including patent applications and publications, are incorporated by reference in their entirety.

### BRIEF SUMMARY

In some aspects, provided herein is a sample collection device comprising an elongated tube body comprising an internal chamber, wherein the elongated tube body comprises a side wall portion, a top portion, and a bottom portion, wherein the elongated tube body has an interior surface and an exterior surface, wherein the top portion comprises an inverted lip forming an opening to the internal chamber, and wherein the inverted lip is configured to form a reservoir between at least a portion of the interior surface of the inverted lip and at least a portion of the interior surface of the side wall portion.

In other aspects, provided herein is a sample collection device comprising an elongated tube body comprising an internal chamber, wherein the elongated tube body comprises a side wall portion, a top portion, and a bottom portion having an interior surface and an exterior surface, wherein the interior surface of the bottom portion comprises a slope forming a depression positioned away from a longitudinal central axis of the elongated tube body, and wherein the top portion comprises an opening. In some embodiments, the depression comprises a lowest point positioned away from a longitudinal central axis of the elongated tube body.

In other aspects, provided herein is a sample collection device comprising an elongated tube body comprising an internal chamber, wherein the elongated tube body comprises a side wall portion, an top portion, a bottom portion having an interior surface and an exterior surface, wherein the top portion comprises an inverted lip forming an opening to the internal chamber, wherein the inverted lip is configured to form a reservoir between at least a portion of the interior surface of the inverted lip and at least a portion of the interior surface of the side wall portion, and wherein the interior surface of the bottom portion comprises a slope forming a depression positioned away from a longitudinal central axis of the elongated tube body. In some embodiments, the depression comprises a lowest point positioned away from a longitudinal central axis of the elongated tube body.

In some embodiments, the volume of the reservoir is at least about 0.25 mL. In some embodiments, the volume of the reservoir is about 0.25 mL to about 5 mL. In some embodiments, the volume of the reservoir is based on liquid capacity when the sample collection device is inverted relative to a longitudinal central axis of the elongated tube body.

In some embodiments, the inverted lip and the elongated tube body are monolithic. In some embodiments, the inverted lip and the elongated tube body are separate pieces. In some embodiments, the elongated tube body comprises a first material and the inverted lip comprises a second material. In some embodiments, the first material of the elongated tube body and the second material of the inverted lip are different. In some embodiments, the first material of the elongated tube body and the second material of the inverted lip are the same. In some embodiments, the first material and the second material comprise a thermoformable polymer.

In some embodiments, the side wall portion comprises a feature configured to interface with the inverted lip, wherein the interface between the side wall portion and the inverted lip forms a fluid-tight seal.

In some embodiments, at least a portion of the exterior surface of the inverted lip comprises a taper in the direction of the top portion to the bottom portion. In some embodiments, at least a portion of the exterior surface of the inverted lip is configured to interface with a disposable pipette tip. In some embodiments, the exterior surface of the inverted lip is a substantially smooth surface. In some embodiments, the exterior surface of the inverted lip comprises at least one rib extending in the direction of a longitudinal central axis of the elongated tube body.

In some embodiments, the opening of the top portion has a largest cross-sectional dimension that is equal to or less than the largest cross-sectional dimension of the internal surface of the side wall portion, wherein the cross-sectional dimension of the opening is substantially perpendicular to a longitudinal central axis of the elongated tube body. In some embodiments, the opening of the top portion has a largest cross-sectional dimension of about 3 mm to about 25 mm. In some embodiments, the opening of the top portion has a largest cross-sectional dimension of about 18 mm or less.

In some embodiments, the ratio between the opening of the top portion and the largest cross-sectional dimension of the internal surface of the side wall portion is about 8:10 to about 1:10.

In some embodiments, the ratio between the height of the elongated tube body and the largest cross-sectional dimension, as measured perpendicular to a longitudinal central axis of the elongated tube body, of the internal surface of the side wall portion is at least about 3:1 to about 20:1.

In some embodiments, the ratio between the opening of the top portion and the height of the inverted lip is at least about 1:10 to about 10:1.

In some embodiments, the ratio between the opening of the elongated tube body formed by the inverted lip and the largest cross-sectional dimensional, as measured perpendicular to a longitudinal central axis of the elongated tube body, of the internal surface of the sidewall portion is about 8:10 to about 1:10.

In some embodiments, the top portion and the bottom portion are configured such that a disposable pipette tip will not contact the interior surface of the bottom portion when the disposable pipette tip is inserted in the opening of the top portion.

In some embodiments, at least a portion of the exterior surface of the side wall portion is cylindrical.

In some embodiments, the length of the elongated tube body is about 2 cm to about 10 cm, as measured parallel to a longitudinal central axis of the elongated tube body.

In some embodiments, a cross-section of the exterior surface of the side wall portion is circular.

In some embodiments, at least a portion of the exterior surface of the bottom portion of the elongated tube body forms a substantially flat bottom relative to a plane perpendicular to a longitudinal central axis of the elongated tube body.

In some embodiments, the internal chamber has a volume of at least about 1.5 mL.

In some embodiments, the sample collection device further comprises a cap. In some embodiments, the cap is a threaded cap. In some embodiments, the exterior surface of the elongated tube body comprises a thread configured to interface with the threaded cap. In some embodiments, the cap is a push cap. In some embodiments, the exterior surface and/or interior surface of the elongated tube body comprises a feature configured to interface with the push cap. In some embodiments, the cap is configured to interface with at least a portion of the inverted lip. In some embodiments, the cap comprises a feature configured to interface with a cap placement/removal device.

In other aspects, provided herein is a kit comprising any sample collection device described herein, and a collection tool. In some embodiments, the collection tool comprises a sample collection portion, a stem portion, and a handle portion, wherein the sample collection portion is on a distal portion of the collection tool, wherein the handle portion is on proximal portion of the collection tool, and wherein the stem portion connects the sample collection portion and the handle portion. In some embodiments, the collection tool comprises a break point feature configured to break the stem portion at a pre-determined location to separate the sample collection portion and the handle portion. In some embodiments, the collection tool is a collection swab. In some embodiments, the size of the collection tool, or portion thereof, configured to be deposited in the sample collection device is based on the size of an internal chamber of the sample collection device. In some embodiments, the size of the collection tool, or portion thereof, configured to be deposited in the sample collection device is based on the size of an internal chamber of the sample collection device and the position of an inverted lip relative to a bottom portion of an elongated tube body of the sample collection device, such that the collection tool, or portion thereof, configured to be deposited in the sample collection device sits below the inverted lip when in contact with the bottom portion of the elongated tube body. In some embodiments, the kit further comprises instructions for collecting a sample.

In other aspects, provided is a method of collecting a sample, the method comprising: obtaining the sample using a collection tool; and inserting at least a portion of the collection tool in a sample collection device via an opening, wherein the sample collection device is described herein, and wherein the sample collection device is configured using the inverted lip to substantially inhibit spillage of a fluid when the opening of the sample collection device is not sealed by a cap.

In other aspects, provided is a method of collecting a sample, the method comprising: obtaining the sample using a collection tool; and inserting at least a portion of the collection tool in a sample collection device via an opening, wherein the sample collection device is described herein, wherein the sample collection device is configured to accept at least a portion of the collection tool in the depression formed by the interior surface of the bottom portion of the elongated tube body of the sample collection device, and wherein the depression is positioned away from a longitudinal central axis of the elongated tube body.

In other aspects, provided is a method of collecting a sample, the method comprising: obtaining the sample using a collection tool; and inserting at least a portion of the collection tool in a sample collection device via an opening, wherein the sample collection device is described herein, wherein the sample collection device is configured using the inverted lip to substantially inhibit spillage of a fluid when the opening of the sample collection device is not sealed by a cap, wherein the sample collection device is configured to accept at least a portion of the collection tool in the depression formed by the interior surface of the bottom portion of the elongated tube body of the sample collection device, and wherein the depression is positioned away from a longitudinal central axis of the elongated tube body.

In some embodiments, the method further comprises closing the sample collection device using the cap. In some embodiments, the cap comprises a pierceable membrane.

In some embodiments, the method further comprises depositing at least a portion of the collection tool in the sample collection device. In some embodiments, the collection tool, or portion thereof, is configured such that when deposited in the sample collection device the collection tool, or portion thereof, is contained in the internal chamber below the opening and proximal to the bottom portion of the elongated tube body of the sample collection device. In some embodiments, depositing at least a portion of the collection tool comprises exerting a force on the collection tool to break off the portion thereof via a break point feature.

In some embodiments, the collection tool is a collection swab.

In some embodiments, the sample is a biological sample comprising a biological material. In some embodiments, the biological material is selected from the group consisting of a nucleic acid and a polypeptide. In some embodiments, the nucleic acid is an RNA or a DNA. In some embodiments, the sample is of, or derived from, an individual. In some embodiments, the individual is a human.

In some embodiments, obtaining the sample comprises sampling the individual, or a substance derived therefrom, using the collection tool.

In some embodiments, the method further comprises obtaining the sample collection device and collection tool from a point of origination. In some embodiments, the method further comprises shipping the capped sample collection device to a destination. In some embodiments, the destination and the point of origination are the same location.

In other aspects, provided is a method of obtaining at least a portion of a composition comprising a sample contained in a sample collection device, the method comprising: opening the sample collection device containing the composition comprising the sample; and drawing at least a portion of the composition comprising the sample out of the sample collection device via a pipette tip, wherein the sample collection device is described herein and comprises the inverted lip.

In other aspects, provided is a method of obtaining at least a portion of a composition comprising a sample contained in a sample collection device, the method comprising: opening the sample collection device containing at least a portion of a collection tool and the composition comprising the sample; and drawing at least a portion of the composition comprising the sample out of the sample collection device via a pipette tip, wherein the sample collection device is described herein and comprises the depression formed via the interior surface of the bottom portion of the elongated tube body of the sample collection device.

In other aspects, provided is a method of obtaining at least a portion of a composition comprising a sample using a sample collection device, the method comprising: opening the sample collection device containing at least a portion of a collection tool and the composition comprising the sample; and drawing at least a portion of the composition comprising the sample out of the sample collection device via a pipette tip, wherein the sample collection device comprises is described herein and comprises the inverted lip and the depression formed via the interior surface of the bottom portion of the elongated tube body of the sample collection device.

In some embodiments, the collection tool, or the portion thereof, is not removed from the sample collection device prior to drawing at least a portion of the composition comprising the sample out of the sample collection device via the pipette tip.

In some embodiments, the sample collection device is configured such that the collection tool, or the portion thereof, in the sample collection device is positioned to allow insertion of the pipette tip without interference from the collection tool, or the portion thereof.

In some embodiments, the method further comprises inserting the pipette tip into the opening of the sample collection device.

In some embodiments, the composition comprises substantially the same amount of a pre-loaded fluid as in the sample collection device prior to addition of the sample.

In some embodiments, opening the sample collection device comprises removing a cap of the sample collection device. In some embodiments, the cap is completed via a cap placement/removal device. In some embodiments, opening the sample collection device comprises piercing a pierceable membrane of a cap.

In some embodiments, the method further comprises reading a label on the sample collection device. In some embodiments, the label comprises information regarding a characteristic of the composition comprising the sample. In some embodiments, the characteristic is selected from the group consisting of volume, contents, source of the sample, and type of the sample.

In some embodiments, the method further comprises subjecting at least a portion of the composition comprising the sample to a downstream analysis technique. In some embodiments, the downstream analysis technique comprises a purification protocol. In some embodiments, the downstream analysis technique comprises an analyte detection protocol.

In some embodiments, the sample is a biological sample comprising a biological material. In some embodiments, the biological material is selected from the group consisting of a nucleic acid and a polypeptide. In some embodiments, the nucleic acid is an RNA or a DNA. In some embodiments, the sample is of, or derived from, an individual. In some embodiments, the individual is a human.

These and other aspects and advantages of the present disclosure will become apparent from the subsequent detailed description and the appended claims. It is to be understood that one, some, or all of the properties of the various embodiments described herein may be combined to form other embodiments of the present disclosure.

### BRIEF DESCRIPTION OF THE DRAWINGS

FIG. 1 shows a cross-sectional view of an exemplary sample collection device 100 comprising an elongated tube body 102, a threaded cap 140, an inverted lip 112, a skirt 124, and a bottom portion 110 comprising a depression 122 positioned off-center of a longitudinal central axis 130 of the elongated tube body 102.

FIG. 2A shows a cross-sectional view of a portion of an exemplary sample collection device 100 in an inverted position. FIG. 2B shows a cross-sectional view of an exemplary inverted lip 112 of a sample collection device, wherein the inverted lip comprises ribs, e.g., 210. FIG. 2C shows a cross-sectional view of an exemplary inverted lip 112 of a sample collection device, wherein the inverted lip does not comprise a rib. FIG. 2D shows a cross-sectional view of a portion of an exemplary sample collection device 100.

FIGS. 3A and 3B show cross-sectional views of an exemplary sample collection device 100, wherein a collection tool, or a portion thereof, 300 is located in an internal chamber 104 of the sample collection device 100.

FIGS. 4A and 4B show cross-sectional views of an exemplary sample collection device 100, wherein a collection tool 300 is located in an internal chamber 104 of the sample collection device 300, and a disposable pipette tip 400 is inserted into the internal chamber via an opening.

FIG. 5 shows an exterior view of an exemplary sample collection device 100.

### DETAILED DESCRIPTION

Provided herein, in some aspects, is a sample collection device comprising an elongated tube body comprising an internal chamber. In some embodiments, the sample collection device comprises an inverted lip forming an opening to the internal chamber, wherein the inverted lip is configured to form a reservoir between at least a portion of an interior surface of the inverted lip and at least a portion of an interior surface of a side wall portion of the elongate tube body. In some embodiments, the sample collection device comprises an interior surface of a bottom portion of the elongated tube body comprising a slope forming a depression positioned away from a longitudinal central axis of the elongated tube body. The present disclosure is based, at least in part, on the inventors' unique insights and findings regarding the design and configuration of sample collection devices that, e.g., prevent spillage of fluid once loaded in a sample collection device, improve safety of use, enable use of hazardous reagents, facilitate transportation thereof, prevent contamination of non-disposable handling equipment (such as by preventing contact of contaminated surfaces of a sample collection device with non-disposable portions of a pipette), and provide for more efficient processing following sample collection. For example, in some embodiments, the sample collection device, when sent to the consumer/patient, comprises a pre-filled reagent, such as a hazardous reagent. The reservoir and opening of an inverted lip of a top portion of an elongated tube body disclosed herein prevent spillage of such reagents should the consumer/patient tip or otherwise mishandle the sample collection device. Furthermore, the reservoir and opening of an inverted lip of a top portion of an elongated tube body disclosed herein blocks access to the portion of a sample collection tool deposited in the sample collection device, thus preventing any pricks due to the formation of a sharp edge of a portion of a sample collection tool. Processing of the biological sample collected in the sample collection devices described herein is greatly improved as the design of the inverted lip and bottom portion ensure that the sample collection tool is position out of way of a pipette tip and non-disposable parts of a pipette tip cannot be exposed to contaminated portions of a sample. Furthermore, assessment for loss of a reagent during sample collection is obviated. Such advantages facilitate high-throughput analysis, with improved quality and consistency, of biological samples obtained using the sample collection devices described herein.

Thus, in some aspects, provided herein is a sample collection device comprising an elongated tube body comprising an internal chamber, wherein the elongated tube body comprises a side wall portion, a top portion, and a bottom portion, wherein the elongated tube body has an interior surface and an exterior surface, wherein the top portion comprises an inverted lip forming an opening to the internal chamber, and wherein the inverted lip is configured to form a reservoir between at least a portion of the interior surface of the inverted lip and at least a portion of the interior surface of the side wall portion.

In other aspects, provided herein, is a sample collection device comprising an elongated tube body comprising an internal chamber, wherein the elongated tube body comprises a side wall portion, a top portion, and a bottom portion having an interior surface and an exterior surface, wherein the interior surface of the bottom portion comprises a slope forming a depression positioned away from a longitudinal central axis of the elongated tube body, and wherein the top portion comprises an opening. In some embodiments, the depression comprises a lowest point positioned away from a longitudinal central axis of the elongated tube body.

In other aspects, provided herein is a sample collection device comprising an elongated tube body comprising an internal chamber, wherein the elongated tube body comprises a side wall portion, an top portion, a bottom portion having an interior surface and an exterior surface, wherein the top portion comprises an inverted lip forming an opening to the internal chamber, wherein the inverted lip is configured to form a reservoir between at least a portion of the interior surface of the inverted lip and at least a portion of the interior surface of the side wall portion, and wherein the interior surface of the bottom portion comprises a slope forming a depression positioned away from a longitudinal central axis of the elongated tube body. In some embodiments, the depression comprises a lowest point positioned away from a longitudinal central axis of the elongated tube body.

In other aspects, provided herein is a kit comprising any sample collection device described herein, and a collection tool.

In other aspects, provided is a method of collecting a sample, the method comprising: obtaining the sample using a collection tool; and inserting at least a portion of the collection tool in a sample collection device via an opening, wherein the sample collection device is described herein, and wherein the sample collection device is configured using the inverted lip to substantially inhibit spillage of a fluid when the opening of the sample collection device is not sealed by a cap.

In other aspects, provided is a method of collecting a sample, the method comprising: obtaining the sample using a collection tool; and inserting at least a portion of the collection tool in a sample collection device via an opening, wherein the sample collection device is described herein, wherein the sample collection device is configured to accept at least a portion of the collection tool in the depression formed by the interior surface of the bottom portion of the elongated tube body of the sample collection device, and wherein the depression is positioned away from a longitudinal central axis of the elongated tube body.

In other aspects, provided is a method of collecting a sample, the method comprising: obtaining the sample using a collection tool; and inserting at least a portion of the collection tool in a sample collection device via an opening, wherein the sample collection device is described herein, wherein the sample collection device is configured using the inverted lip to substantially inhibit spillage of a fluid when the opening of the sample collection device is not sealed by a cap, wherein the sample collection device is configured to accept at least a portion of the collection tool in the depression formed by the interior surface of the bottom portion of the elongated tube body of the sample collection device, and wherein the depression is positioned away from a longitudinal central axis of the elongated tube body.

In other aspects, provided is a method of obtaining at least a portion of a composition comprising a sample contained in a sample collection device, the method comprising: opening the sample collection device containing the composition comprising the sample; and drawing at least a portion of the composition comprising the sample out of the sample collection device via a pipette tip, wherein the sample collection device is described herein and comprises the inverted lip.

In other aspects, provided is a method of obtaining at least a portion of a composition comprising a sample contained in a sample collection device, the method comprising: opening the sample collection device containing at least a portion of a collection tool and the composition comprising the sample; and drawing at least a portion of the composition comprising the sample out of the sample collection device via a pipette tip, wherein the sample collection device is described herein and comprises the depression formed via the interior surface of the bottom portion of the elongated tube body of the sample collection device.

In other aspects, provided is a method of obtaining at least a portion of a composition comprising a sample using a sample collection device, the method comprising: opening the sample collection device containing at least a portion of a collection tool and the composition comprising the sample; and drawing at least a portion of the composition comprising the sample out of the sample collection device via a pipette tip, wherein the sample collection device comprises is described herein and comprises the inverted lip and the depression formed via the interior surface of the bottom portion of the elongated tube body of the sample collection device.

It will also be understood by those of ordinary skill in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects.

### A. Definitions

As used herein, an “individual” is a mammal. In some embodiments, a “mammal” includes humans, non-human primates, domestic and farm animals, and zoo, sports, or pet animals, such as dogs, horses, rabbits, cattle, pigs, hamsters, gerbils, mice, ferrets, rats, cats, monkeys, etc. In some embodiments, the individual is human.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Those skilled in the art will recognize that several embodiments are possible within the scope and spirit of the present disclosure. The following description illustrates the disclosure and, of course, should not be construed in any way as limiting the scope of the inventions described herein.

### B. Sample Collection Devices

In some aspects, provided herein is a sample collection device. As discussed herein, in some embodiments, the sample collection devices described herein are configured with features to safely and efficiently enable collection and processing of a sample, such as a biological sample.

In some embodiments, the sample device comprises an elongated tube body comprising an internal chamber, wherein the elongated tube body comprises a side wall portion, a top portion, and a bottom portion, and wherein the elongated tube body has an interior surface and an exterior surface. As described herein, the term “elongated,” in “elongated tube body,” is used to describe the relationship between the height of the elongated tube body and the largest cross-sectional dimension of an exterior surface of the elongated tube body (as measured perpendicular to a longitudinal central axis of the elongated tube body). For example, in some embodiments, the elongated tube body has a ratio of height (such as measured when the elongated tube is standing on a horizontal surface) to largest cross-sectional dimension of an exterior surface of the elongated tube body (as measured perpendicular to a longitudinal central axis of the elongated tube body; e.g., diameter) of at least about 2:1, such as at least about any of 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, or 10:1. In some embodiments, the sample device comprises a top portion comprising an inverted lip forming an opening to an internal chamber, and wherein the inverted lip is configured to form a reservoir between at least a portion of an interior surface of the inverted lip and at least a portion of an interior surface of a side wall portion. In some embodiments, the sample device comprises an interior surface of a bottom portion comprising a slope forming a depression positioned away from a longitudinal central axis of the elongated tube body, and wherein the top portion comprises an opening. In some embodiments, the depression comprises a lowest point positioned away from a longitudinal central axis of the elongated tube body. In some embodiments, the sample device comprises a top portion comprising an inverted lip forming an opening to an internal chamber, wherein the inverted lip is configured to form a reservoir between at least a portion of an interior surface of the inverted lip and at least a portion of an interior surface of a side wall portion, and the sample device comprises an interior surface of a bottom portion comprising a slope forming a depression positioned away from a longitudinal central axis of the elongated tube body, and wherein the top portion comprises an opening. In some embodiments, the depression comprises a lowest point positioned away from a longitudinal central axis of the elongated tube body.

The sample collection devices described herein are useful for collecting a diverse array of samples. In some embodiments, the sample is a biological sample comprising a biological material. In some embodiments, the biological material is selected from the group consisting of a nucleic acid and a polypeptide. In some embodiments, the nucleic acid is an RNA or a DNA. In some embodiments, the biological sample is of, or derived from, an individual, such as a human. In some embodiments, the sample, such as the biological sample, is an environmental sample. In some embodiments, the sample, such as the biological sample, comprises a synthetic material, such as a man-made material.

For purposes of illustrating exemplary sample collection devices and features thereof, a description of certain figures is provided below. Such description is not intended to limit the scope of the sample collection devices described herein.

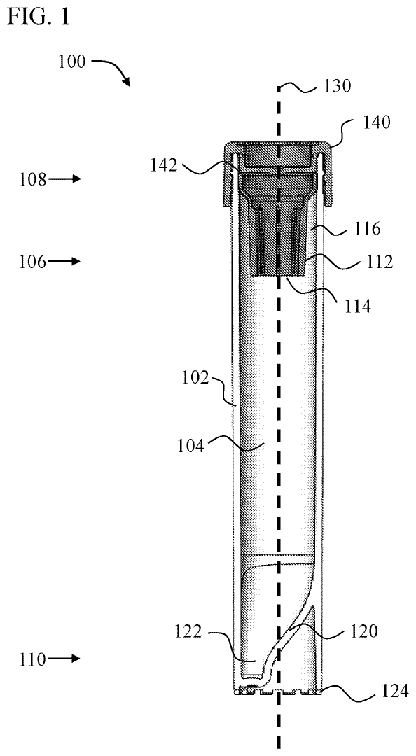

An exemplary sample collection device 100 is illustrated in cross-sectional diagram of FIG. 1. As shown in FIG. 1, the sample collection device 100 comprises an elongated tube body 102 comprising an internal chamber 104. The elongated tube body comprises a side wall portion 106, a top portion 108, and a bottom portion 110. The terms of the side wall portion, the top portion, and the bottom portion are provided as useful descriptors and the boundaries between the various portions of the elongated tube body can have a certain amount of flexibility according to the description of the various features of the sample collection device provided herein. As illustrated, the elongated tube body 102 has an interior surface, e.g., facing the internal chamber 104, and an exterior surface, e.g., facing the environment outside of the sample collection device 100. In some embodiments, the sample collection device 100 comprises a top portion 108 comprising an inverted lip 112 forming an opening 114 to the internal chamber 104. The inverted lip 112 is configured to form a reservoir 116 between at least a portion of the interior surface of the inverted lip 112 and at least a portion of the interior surface of the side wall portion 106.

As shown in FIG. 1, in some embodiments, the sample collection device comprises an interior surface of a bottom portion 110 comprises a slope 120 forming a depression 122 having a lowest point positioned away from a longitudinal central axis 130 of the elongated tube body 102. As illustrated, the sample collection device 100 comprises at least a portion of the exterior surface of the bottom portion 110 of the elongated tube body 102 having a substantially flat bottom relative to a plane perpendicular to a longitudinal central axis of the elongated tube body. In some embodiments, the portion of the bottom portion 110 of the elongated tube body 102 having a substantially flat bottom is in the form of a skirt 124.

As shown in FIG. 1, in some embodiments, the sample collection device comprises a cap 140. In some embodiments, the cap 140 is a screw cap that interfaces with threads on the exterior surface of the elongate tube body 102. In some embodiments, the cap 140 is configured to interface with an inverted lip 112, such as shown at 142. In some embodiments, the action of placing the cap 140 on the elongated tube body 102, such as by screwing the cap down, seats the inverted lip 112 in a pre-determined configuration.

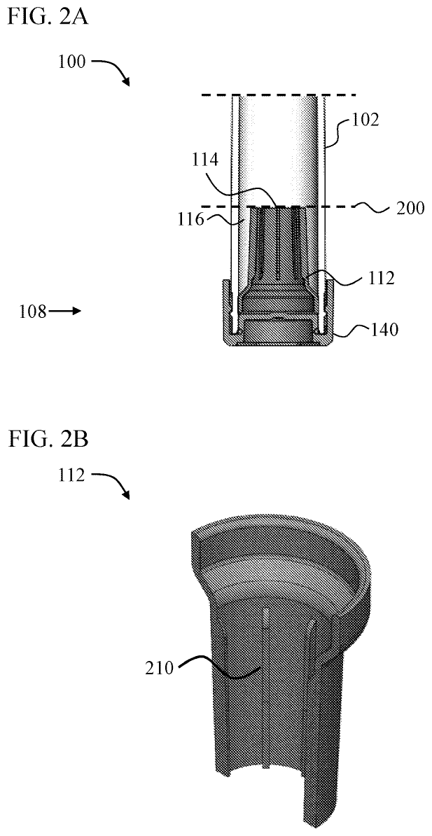

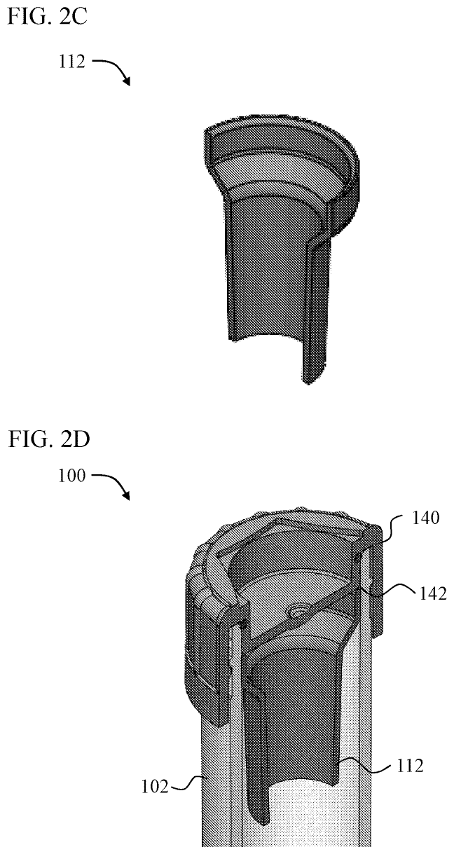

FIG. 2A provides a cross-sectional illustration of a portion of the sample collection device 100 inverted (180°) relative to the longitudinal central axis of the elongated tube body 130 to demonstrate the liquid capacity of a reservoir 116. As illustrated by the dashed line 200, upon inversion of the sample collection device, the reservoir 116 can hold, e.g., a fluid based upon the volume of the reservoir 116 formed by the interior surface of the inverter lip 112 and the interior surface of the side wall, such that fluid can reside in the reservoir 116 to about the level of the dashed line 200 before it will drain from the opening 114 formed by the inverted lip 112.

FIG. 2B provides a magnified cross-sectional view of an inverted lip 112. As shown in FIG. 2B, in some embodiments, the exterior surface of the inverted lip comprises at least one rib 210 extending in the direction of a longitudinal central axis of the elongated tube body. In some embodiments, the exterior surface of the inverted lip is a substantially smooth surface (not shown in FIG. 2B).

FIG. 2C provides a magnified cross-sectional view of an inverted lip 112. As shown in FIG. 2C, in some embodiments, the exterior surface of the inverted lip does not comprise a rib (e.g., is a smooth surface).

FIG. 2D provides a cross-sectional illustration of a portion of the sample collection device 100, wherein the inverted lip 112 of the top portion of the elongated tube body 102 does not comprise a rib (e.g., is a smooth surface). FIG. 2D illustrates the placements of a cap 140 configured to interface with the inverted lip 112, such as shown at 142.

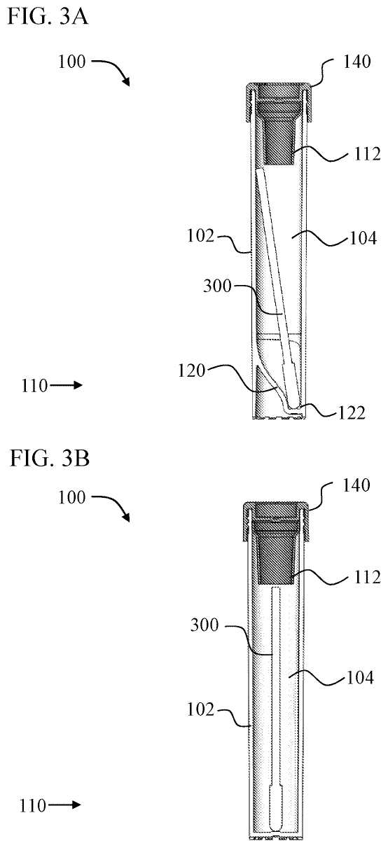

In some embodiments, the bottom portion of a sample collection device is configured to interface with a collection tool. For example, as shown in FIG. 3A, in some embodiments, the sample collection device comprises an interior surface of a bottom portion 110 comprising a slope 120 forming a depression 122 having a lowest point positioned away from a longitudinal central axis of the elongated tube body. The bottom portion 110 is configured to interface with a collection tool 300, such that, e.g., the collection tool is positioned to allow sampling of a fluid from the opening and the collection tool displaces fluid from the depression 122 to improve retrieval yield. An alternative view of the positioning of the collection tool 300 in the sample collection device 100 is provided in FIG. 3B. As discussed herein and illustrated in FIG. 3B, the sizing and design of the sample collection device 100, and features thereof and/or placed therein, can be based on various needs and/or constraints, such as the size of a portion of the collection tool 300 deposited in the sample collection device 100.

As shown in FIGS. 3A and 3B, the collection tool, or portion thereof 300, is configured such that when deposited in the sample collection device 100 (such as fully inserted) the entire collection tool, or entire portion thereof, is contained in the internal chamber 104 below the opening of the inverted lip 112 and proximal to the bottom portion 110 of the elongated tube body 102 of the sample collection device 100.

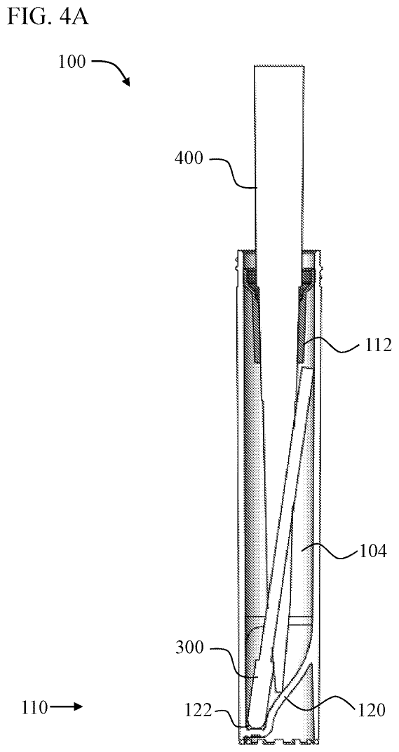

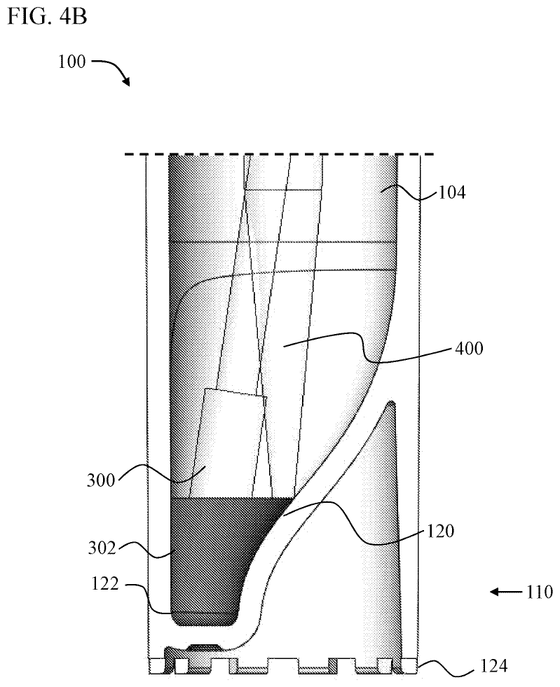

In some aspects, the sample collection devices described herein are configured to interface with a pipette tip, such as a disposable pipette tip. For example, as shown in FIG. 4A, the sample collection device 100 comprises an inverted lip 112 configured with an opening to accept a disposable pipette tip 400. The bottom portion 110 of the sample collection device 100 comprises a depression 122 configured to interface with a collection tool 300, such that the collection tool 300 is positioned within the sample collection device 100 to allow for the disposable pipette tip 400 to access a fluid without interference from the collection tool 300. Such configuration enables the disposable pipette tip 400 to freely travel in a direction, such as along the longitudinal central axis of the elongated tube body, and to draw fluid into the disposable pipette tip 400 with without getting clogged by a portion of the collection tool 300. A magnified cross-sectional view of an exemplary sample collection device 100 is provided in FIG. 4B to illustrate the bottom portion 110 having a depression 122 configured to interface with a collection tool 300, such that the collection tool 300 is positioned within the sample collection device 100 to allow for the disposable pipette tip 400 to access a fluid 302 without interference from the collection tool 300.

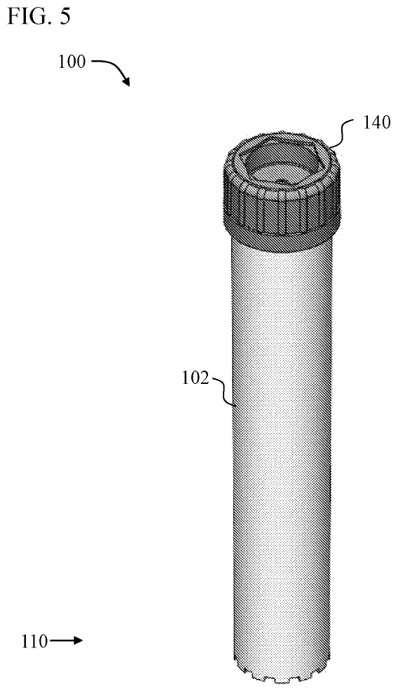

FIG. 5 illustrates an external view of the sample collection device 100. As illustrated, the sample collection device 100 comprises an elongated tube body 102 and a cap 140. In some embodiments, the cap 140 comprises one or more features configured to interface with a cap placement/removal device and/or ease the placement and removal of the cap 140. As shown in FIG. 5, shapes, depressions, and protrusions may be used on the cap 140. In some embodiments, the bottom portion 110 of the sample collection device 100 comprises one or more features configured to interface with another device for holding the sample collection device 100. For example, as shown in FIG. 5, the bottom portion 110 comprises one or more notches formed by a skirt 124. In some embodiments, the one or more notches hold the elongated tube body of the sample collection device 100 stationary when a force, such as torque, is applied to a cap of the sample collection device 100.

Certain configurations and features of the sample collection devices are described in additional detail in the sections below. The modular discussion of certain configurations and features is not intended to limit the scope of the sample collection devices described herein, and using the teachings provided herein one can readily combine various modularly described configurations and features to arrive at the full scope of the sample collection devices provided herein.

i. Inverted Lips and Reservoirs

In some embodiments, the sample collection device comprises an inverted lip that forms a reservoir in an internal chamber of an elongated tube body. As described and illustrated herein, in some embodiments, a top portion of an elongated tube body comprises the inverted lip forming an opening to the internal chamber, wherein the inverted lip is configured to form a reservoir between at least a portion of an interior surface of the inverted lip and at least a portion of an interior surface of the side wall portion of the elongate tube body.

In some embodiments, the volume of a reservoir is based on the volume of a component added to an internal chamber of a sample collection device, e.g., a fluid such as a sample processing buffer that pre-fills the sample collection device prior to sample collection and/or a fluid and an expected sample volume. In some embodiments, the volume of a reservoir is at least about 0.2 mL, such as at least about any of 0.3 mL, 0.4 mL, 0.5 mL, 0.6 mL, 0.7 mL, 0.8 mL, 0.9 mL, 1 mL, 1.1 mL, 1.2 mL, 1.3 mL, 1.4 mL, 1.5 mL, 1.6 mL, 1.7 mL, 1.8 mL, 1.9 mL, 2 mL, 2.1 mL, 2.2 mL, 2.3 mL, 2.4 mL, 2.5 mL, 2.6 mL, 2.7 mL, 2.8 mL, 2.9 mL, 3 mL, 3.1 mL, 3.2 mL, 3.3 mL, 3.4 mL, 3.5 mL, 3.6 mL, 3.7 mL, 3.8 mL, 3.9 mL, 4 mL, 4.1 mL, 4.2 mL, 4.3 mL, 4.4 mL, 4.5 mL, 4.6 mL, 4.7 mL, 4.8 mL, 4.9 mL, or 5 mL. In some embodiments, the volume of a reservoir is about 0.25 mL to about 5 mL, such as about 0.75 mL to about 2 mL or about 1 mL to about 1.5 mL. As described herein, in some embodiments, the volume of a reservoir is based on the amount of liquid the reservoir can hold (without spillage of the liquid through the opening) when a sample collection device is inverted relative to a longitudinal central axis of an elongated tube body of the sample collection device (such as inverted 180° as compared to when the sample collection device is place upright on a horizontal surface and standing via the bottom portion of the elongated tube body).

In some embodiments, the ratio between the length of the inverted lip that contributes to the formation of the reservoir and the opening of the top portion is at least about 1:2 and at most about 10:1.

The inverted lip may be constructed using a number of different approaches and using a variety of materials. In some embodiments, the inverted lip and the elongated tube body are monolithic (i.e., formed as a single piece). In some embodiments, the inverted lip and the elongated tube body are separate pieces. In some embodiments, when the inverted lip and the elongated tube body are separate pieces, the side wall portion of the elongated tube body comprises a feature configured to interface with the inverted lip, wherein the interface between the side wall portion and the inverted lip forms a fluid-tight seal. In some embodiments, when the inverted lip and the elongated tube body are separate pieces, the inverted lip comprises a feature configured to interface with the side wall portion of the elongated tube body, wherein the interface between the side wall portion and the inverted lip forms a fluid-tight seal. In some embodiments, when the inverted lip and the elongated tube body are separate pieces, the side wall portion of the elongated tube body and the inverted lip comprise one or more features configured to interface with one another, wherein the interface between the side wall portion and the inverted lip forms a fluid-tight seal. In some embodiments, the feature configured to interface the inverted lip and the elongated tube body comprises any one or more of a protrusion, depression, or size and/or shape restriction. In some embodiments, the sample collection device is configured such that an inverted lip is a separate piece than an elongated tube body, and the inverted lip can be seated into a pre-determined position, e.g., such as by force of the applying a cap to the sample collection device.

In some embodiments, the elongated tube body comprises a first material and an inverted lip comprises a second material. In some embodiments, the first material of the elongated tube body and the second material of the inverted lip are different. In some embodiments, the first material of the elongated tube body and the second material of the inverted lip are the same. In some embodiments, the first material and the second material are a thermoformable polymer. In some embodiments, the first material and the second material are plastic. In some embodiments, the first material and the second material are substantially devoid of a nucleic acid, such as a DNA or RNA. In some embodiments, the first material and the second material resist binding of a nucleic acid, such as a DNA or RNA. In some embodiments, the first material and/or the second material are substantially resistant to degradation from solvents (e.g., alcohol compounds) and other chemicals/solutions/reagents (e.g., highly basic or acidic salt solutions).

In some embodiments, at least a portion of the exterior surface of an inverted lip comprises a taper in the direction of a top portion to a bottom portion of the elongated tube body. In some embodiments, at least a portion of the exterior surface of an inverted lip is configured to interface with a pipette tip, such as a disposable pipette tip. In some embodiments, the exterior surface of an inverted lip is a substantially smooth surface.

In some embodiments, the exterior surface of an inverted lip comprises at least one feature to prevent a pipette tip from sticking to and/or sealing with the inverted lip of a sample collection device. In some embodiments, the exterior surface of an inverted lip comprises between 3 and 24 ribs. In some embodiments, the exterior surface of an inverted lip comprises at least 3, such as at least any of 4, 5, 6, 7, 8, 9, 10, 11, 12, 13, 14, 15, 16, 17, 18, 19, 20, 21, 22, 23, or 24, ribs. In some embodiments, the exterior surface of an inverted lip comprises at least 3, such as at least any of 4, 5, 6, 7, 8, 9, 10, 11, 12, 13, 14, 15, 16, 17, 18, 19, 20, 21, 22, 23, or 24, ribs extending in the direction of a longitudinal central axis of the elongated tube body.

The opening to an internal chamber of a sample collection device can be a variety of sizes and shapes. In some embodiments, the opening has a largest cross-sectional dimension based on a minimum size requirement, e.g., the minimum size needed accept a sample collection tool or portion thereof. In some embodiments, the opening of a top portion (such as formed by an inverted lip) of an elongated tube body has a largest cross-sectional dimension that is equal to or less than the largest cross-sectional dimension of an internal surface of a side wall portion of the elongated tube body, wherein the cross-sectional dimension of the opening is substantially perpendicular to a longitudinal central axis of the elongated tube body. In some embodiments, the opening of a top portion (such as formed by an inverted lip) of an elongated tube body has a largest cross-sectional dimension of about 3 mm to about 25 mm, such as about 10 mm to about 18 mm. In some embodiments, the opening of a top portion (such as formed by an inverted lip) has a largest cross-sectional dimension of about 25 mm or less, such as about any of 24 mm or less, 23 mm or less, 22 mm or less, 21 mm or less, 20 mm or less, 19 mm or less, 18 mm or less, 17 mm or less, 16 mm or less, 15 mm or less, 14 mm or less, 13 mm or less, 12 mm or less, 11 mm or less, 10 mm or less, 9 mm or less, 8 mm or less, 7 mm or less, 6 mm or less, 5 mm or less, 4 mm or less, or 3 mm or less.

ii. Bottom Portions

In some embodiments, the sample collection device comprises a bottom portion of an elongated tube body, wherein the bottom portion comprises an interior surface having a slope forming a depression positioned away from a longitudinal central axis of the elongated tube body. In some embodiments, the depression has a lowest point position away from a longitudinal central axis of an elongated tube body.

In some embodiments, the depression comprises a size and/or shape configured to accept at least a portion of a sample collection tool, such as a collection swab. In some embodiments, the bottom portion is configured to position a sample collection tool in a desired configuration when the sample collection tool is deposited in a sample collection device.

In some embodiments, the bottom portion of an elongated tube body is configured relative to a top portion of the elongated tube body such that a pipette tip will contact (or be positioned relative to) an interior surface of the bottom portion when the pipette tip is inserted in the opening of the top portion.

In some embodiments, the bottom portion of an elongated tube body is configured such that a sample collection device can independently stand on a substantially horizontal surface (such as a counter or table). In some embodiments, at least a portion of the exterior surface of a bottom portion of an elongated tube body forms a substantially flat bottom relative to a plane perpendicular to a longitudinal central axis of the elongated tube body.

iii. Size, Construction, and Additional Features of the Sample Collection Devices

The sample collection devices described herein can be formed in various sizes, shapes, and configurations. In some embodiments, the size and/or shape and/or configuration of a sample collection device is based on a desired attribute (e.g., the volume of fluid desired to be held in an internal chamber and/or reservoir) and/or characteristics for interfacing with a component, another device, and/or workflow (e.g., size/shape of a collection tool deposited in the sample collection device and/or size/shape of pipette tip and/or interface with an automated pipetting system and/or interface with a collection sample device rack system).

In some embodiments, the elongated tube body of a sample processing device comprises a first material. The sample collection devices disclosed herein may be constructed from materials suitable for an intended use, such as based on the sample to be obtained and/or the compositions that will be introduced to the sample collection device. In some embodiments, the first material is a thermoformable polymer. In some embodiments, the first material is plastic. In some embodiments, the first material is substantially devoid of a nucleic acid, such as a DNA or RNA. In some embodiments, the first material resists binding of a nucleic acid, such as a DNA or RNA. In some embodiments, the first material is substantially resistant to degradation from solvents (e.g., alcohol compounds) and other chemicals/solutions/reagents (e.g., highly basic or acidic salt solutions).

In some embodiments, at least a portion of the exterior surface of a side wall portion of an elongated tube body is cylindrical. In some embodiments, the exterior surface of a side wall portion of an elongated tube body comprises an elliptic cylinder. In some embodiments, the exterior surface of a side wall portion of an elongated tube body comprises a rectangular prism. In some embodiments, the cross-section of an exterior surface of a side wall portion is circular. In some embodiments, the cross-section of an exterior surface of a side wall portion is square or rectangular. In some embodiments, the cross-section of an exterior surface of a side wall portion is elliptical. In some embodiments, the cross-section of the exterior surface is assessed on a plane perpendicular to a longitudinal central axis of an elongated tube body.

In some embodiments, the length of the elongated tube body, as measured parallel to a longitudinal central axis of the elongated tube body, is about 2 cm to about 20 cm, such as any of about 2 cm to about 15 cm, or about 5 cm to about 10 cm. In some embodiments, the length of the elongated tube body, as measured parallel to a longitudinal central axis of the elongated tube body, is less than about 20 cm, such as less than about any of 19 cm, 18 cm, 17 cm, 16 cm, 15 cm, 14 cm, 13 cm, 12 cm, 11 cm, 10 cm, 9 cm, 8 cm, 7 cm, 6 cm, 5 cm, 4 cm, or 3 cm.

In some embodiments, the internal chamber of an elongated tube body has a volume of about 0.5 mL to about 100 mL, such as any of about 1 mL to about 10 mL or about 1 mL to about 5 mL. In some embodiments, the internal chamber of an elongated tube body has a volume of at least about 1.5 mL, such as at least about any of 2 mL, 2.5 mL, 3 mL, 3.5 mL, 4 mL, 4.5 mL, 5 mL, 5.5 mL, 6 mL, 6.5 mL, 7 mL, 7.5 mL, 8 mL, 8.5 mL, 9 mL, 9.5 mL, or 10 mL.

In some embodiments, the dimensions of the internal chamber of an elongated tube body are configured relative to a dimension of a sample collection tool. In some embodiments, the dimensions of the internal chamber of an elongated tube body include a linear distance that is at least about 1 mm larger than the largest dimension of the of a sample collection tool, or portion thereof, deposited in a sample collection device (e.g., from the bottom of a depression of a bottom portion to a point of the inverted lip closest to the bottom portion).

In some embodiments, the height of an elongated tube body (such as measured when the elongated tube is standing on a horizontal surface) is configured relative to an opening diameter of said elongated tube body. In some embodiments, the ratio between the height of the elongated tube and the opening diameter is at least about 3:1 and at most about 20:1.

In some embodiments, the largest dimension (e.g., diameter) of an opening of the elongated tube body formed by an inverted lip is configured such that the ratio between the largest dimension of the opening and a largest cross-sectional dimension (as assessed perpendicular to a longitudinal central axis of the elongated tube body) of an internal surface of a sidewall portion is at most about 8:10 and at least about 1:10.

In some embodiments, the sample collection device comprises an identifying feature. In some embodiments, the identifying feature is a label. In some embodiments, the label is a scannable label, such as a barcode or QR code. In some embodiments, the identifying feature comprises information regarding any one or more of the following: source, contents, associated patient, processing steps, age, or mailing date.

The sample collection devices, and components thereof, can be manufactured via a number of techniques available in the art. In some embodiments, the manufacturing technique comprises an injection molding technique. In some embodiments, the manufacturing technique comprises a three-dimensional printing technique. In some aspects, provided herein, are methods of manufacturing any sample collection device described herein, the methods comprising forming the sample collection device using a manufacturing technique.

iv. Caps

In some aspects, provided herein is a cap configured to interface with a sample collection device. In some embodiments, the cap is configured to interface with at least a portion of an inverted lip. In some embodiments, the cap is configured to seal an internal chamber of an elongated tube body. In some embodiments, the cap is configured to seat (such as in a pre-determined position) an inverted lip.

In some embodiments, the cap is a threaded cap. In some embodiments, when the cap is a threaded cap, the exterior surface of an elongated tube body comprises a thread configured to interface with the threaded cap.

In some embodiments, the cap is a push cap. In some embodiments, when the cap is a push cap, the exterior surface and/or interior surface of an elongated tube body comprises a feature configured to interface with the push cap. In some embodiments, the feature of configured to interface with the push cap is any one or more of a protrusion, depression, or size and/or shape restriction.

In some embodiments, the cap comprises a pierceable membrane. In some embodiments, the pierceable membrane is configured such that the internal chamber can be accessed from outside a sample collection device via the pierceable membrane. In some embodiments, the pierceable membrane can be pierced using a pipette tip, such as a disposible pipette tip. In some embodiments, the pierceable membrane can be pierced using a needle. In some embodiments, the pierceable membrane comprises a material selected from the group consisting of a foil, thermoplastic material, silicon, paper, and wax.

In some embodiments the cap comprises a feature configured to interface with a cap placement/removal device. In some embodiments, the exterior surface of a cap comprises any one or more of a shape, depressions, or protrusions.

v. Exemplary Devices

In some aspects, provided is a sample collection device comprising an elongated tube body comprising an internal chamber, wherein the elongated tube body comprises a side wall portion, a top portion, and a bottom portion, wherein the elongated tube body has an interior surface and an exterior surface, wherein at least a portion of the exterior surface of the side wall portion of the elongated tube body is cylindrical, wherein the height of the elongated tube body (such as measured when the elongated tube body is standing on a horizontal surface) is about 105 mm to about 110 mm, such as about 108 mm, wherein the largest cross-sectional dimension of the exterior surface of the elongated tube body (as measured perpendicular to a longitudinal central axis of the elongated tube body) is about 15 mm to about 20 mm, such as about 18 mm, wherein the top portion comprises an inverted lip forming an opening to the internal chamber, wherein the inverted lip is configured to form a reservoir between at least a portion of the interior surface of the inverted lip and at least a portion of the interior surface of the side wall portion, wherein the volume of the reservoir is about 1 mL to about 2 mL, wherein the inverted lip and the elongated tube body are formed as separate pieces, and when the inverted lip and the elongated tube body are properly joined the interface between the side wall portion of the elongated tube body and the inverted lip forms a fluid-tight seal, wherein the opening of the inverted lip has is about 5 mm to about 10 mm, such as about 8 mm, wherein at least a portion of the exterior surface of the inverted lip comprises a taper in the direction of the top portion to the bottom portion of the elongated tube body, wherein the exterior surface of the top portion of the elongated tube body comprises a thread configured to interface with a threaded cap, and wherein the bottom portion of the elongated tube body comprises as skirt forming a substantially flat bottom. In some embodiments, the sample collection device further comprises a threaded cap.

### C. Kits and Components of, or for Use with, the Sample Collection Device, and Compositions Thereof

In other aspects, provided herein are kits and components of, or for use with, any of the sample collection devices described herein.

In some embodiments, provided is a kit comprising any sample collection device described herein, and a collection tool. In some embodiments, the collection tool comprises a sample collection portion, a stem portion, and a handle portion, wherein the sample collection portion is on a distal portion of the collection tool, wherein the handle portion is on proximal portion of the collection tool, and wherein the stem portion connects the sample collection portion and the handle portion. In some embodiments, the collection tool comprises a break point feature configured to break the stem portion at a pre-determined location to separate the sample collection portion and the handle portion. In some embodiments, the collection tool is a collection swab. In some embodiments, the sample collection portion of a sample collection tool comprises any one or more of a fibrous material, a porous material, bristles, or a scoop and/or scraping feature.

In some embodiments, the kit compromises a collection tool that, when in its final state (e.g., broken at a breakpoint by a user) fits within a sample collection device. In some embodiments, when the sample collection device comprises an inverted lip via a top portion of an elongated tube body, the collection tool fits within the sample collection device and below the inverted lip. In some embodiments, the size of the collection tool, or portion thereof, configured to be deposited in the sample collection device is based on the size of an internal chamber of the sample collection device and the position of an inverted lip relative to a bottom portion of an elongated tube body of the sample collection device, such that the collection tool, or portion thereof, configured to be deposited in the sample collection device sits below the inverted lip when in contact with the bottom portion of the elongated tube body.

In some embodiments, the kit comprises a reagent, e.g., a sample processing buffer, such as to preserve or prepare the collected sample for downstream application. In some embodiments, the reagent comprises an anti-infectious agent composition, such as a denaturing and/or inhibiting composition. In some embodiments, the reagent is contained in an internal chamber of a sample collection device of the kit.

In some embodiments, the kit further comprises instructions for collecting a sample.

In other aspects, provided herein are compositions comprising a biological sample and a reagent contained in a sample collection device described herein, wherein the reagent volume is a pre-determined volume and no reagent has been lost to spillage. In some embodiments, the composition further comprises a collection tool, or portion thereof. In some embodiments, the composition further comprises a cap.

### D. Methods of Using a Sample Collection Device

In other aspects, provided herein are methods enabled by the sample collection devices described herein.

In some embodiments, provided is a method of collecting a sample, the method comprising: obtaining the sample using a collection tool; and inserting at least a portion of the collection tool in a sample collection device via an opening, wherein the sample collection device is configured using the inverted lip to substantially inhibit spillage of a fluid when the opening of the sample collection device is not sealed by a cap. In some embodiments, the method further comprises closing the sample collection device using a cap. In some embodiments, the cap comprises a pierceable membrane. In some embodiments, the fluid is a pre-loaded fluid, such as a reagent, that is loaded in the sample collection device prior to depositing a sample. In some embodiments, the pre-loaded fluid is loaded in the sample collection device by a first individual, and a second individual collects the sample. In some embodiments, the inverted lip substantially inhibits spillage of a fluid when the sample collection device is capped. In some embodiments, an inverted lip that substantially inhibits spillage refers to a configuration that inhibits less than about 10%, such as less than about any of 9%, 8%, 7%, 6%, 5%, 4%, 3%, 2%, or 1%, loss of the fluid via the opening.

In some embodiments, provided is a method of collecting a sample, the method comprising: obtaining the sample using a collection tool; and inserting at least a portion of the collection tool in a sample collection device via an opening, wherein the sample collection device is configured to accept at least a portion of the collection tool in the depression formed by the interior surface of the bottom portion of the elongated tube body of the sample collection device, and wherein the depression is positioned away from a longitudinal central axis of the elongated tube body. In some embodiments, the method further comprises closing the sample collection device using a cap. In some embodiments, the cap comprises a pierceable membrane. In some embodiments, the fluid is a pre-loaded fluid, such as a reagent, that is loaded in the sample collection device prior to depositing a sample. In some embodiments, the pre-loaded fluid is loaded in the sample collection device by a first individual, and a second individual collects the sample.

In some embodiments, provided is a method of collecting a sample, the method comprising: obtaining the sample using a collection tool; and inserting at least a portion of the collection tool in a sample collection device via an opening, wherein the sample collection device is configured using the inverted lip to substantially inhibit spillage of a fluid when the opening of the sample collection device is not sealed by a cap, wherein the sample collection device is configured to accept at least a portion of the collection tool in the depression formed by the interior surface of the bottom portion of the elongated tube body of the sample collection device, and wherein the depression is positioned away from a longitudinal central axis of the elongated tube body. In some embodiments, the method further comprises closing the sample collection device using a cap. In some embodiments, the cap comprises a pierceable membrane. In some embodiments, the fluid is a pre-loaded fluid, such as a reagent, that is loaded in the sample collection device prior to depositing a sample. In some embodiments, the pre-loaded fluid is loaded in the sample collection device by a first individual, and a second individual collects the sample. In some embodiments, the inverted lip substantially inhibits spillage of a fluid when the sample collection device is capped. In some embodiments, an inverted lip that substantially inhibits spillage refers to a configuration that inhibits less than about 10%, such as less than about any of 9%, 8%, 7%, 6%, 5%, 4%, 3%, 2%, or 1%, loss of the fluid via the opening.

In some embodiments, the method further comprises depositing at least a portion of the collection tool in the sample collection device. In some embodiments, depositing at least a portion of the collection tool comprises exerting a force on the collection tool to break off the portion thereof via a break point feature. In some embodiments, the collection tool, or portion thereof, is configured such that when deposited in the sample collection device the collection tool, or portion thereof, is contained in the internal chamber below the opening and proximal to the bottom portion of the elongated tube body of the sample collection device. In some embodiments, the opening forms an imaginary plane, such as perpendicular to a longitudinal central axis of an elongated tube body, wherein the collection tool, or portion thereof, is configured such that when deposited in the sample collection device the collection tool, or portion thereof, is contained in the internal chamber below the imaginary plane and proximal to the bottom portion of the elongated tube body of the sample collection device.

In some embodiments, the collection tool is a collection swab.

In some embodiments, the sample is a biological sample comprising a biological material. In some embodiments, the biological material is selected from the group consisting of a nucleic acid and a polypeptide. In some embodiments, the nucleic acid is an RNA or a DNA.

In some embodiments, the sample is of, or derived from, an individual. In some embodiments, the individual is a human.

In some embodiments, obtaining the sample comprises sampling the individual, or a substance derived therefrom, using the collection tool.

In some embodiments, the method further comprises obtaining the sample collection device and collection tool from a point of origination. In some embodiments, the method further comprises shipping the capped sample collection device to a destination. In some embodiments, the destination and the point of origination are the same location.

In some embodiments, provided is a method of obtaining at least a portion of a composition comprising a sample contained in a sample collection device, the method comprising: opening the sample collection device containing the composition comprising the sample; and drawing at least a portion of the composition comprising the sample out of the sample collection device via a pipette tip, wherein the sample collection device is described herein and comprises the inverted lip. In some embodiments, the collection tool, or the portion thereof, is not removed from the sample collection device prior to drawing at least a portion of the composition comprising the sample out of the sample collection device via the pipette tip. In some embodiments, the method further comprises inserting the pipette tip into the opening of the sample collection device. In some embodiments, inserting a pipette tip into the opening of the sample collection device does not contaminate a non-disposable portion of a pipette.

In some embodiments, provided is a method of obtaining at least a portion of a composition comprising a sample contained in a sample collection device, the method comprising: opening the sample collection device containing at least a portion of a collection tool and the composition comprising the sample; and drawing at least a portion of the composition comprising the sample out of the sample collection device via a pipette tip, wherein the sample collection device is described herein and comprises the depression formed via the interior surface of the bottom portion of the elongated tube body of the sample collection device. In some embodiments, the collection tool, or the portion thereof, is not removed from the sample collection device prior to drawing at least a portion of the composition comprising the sample out of the sample collection device via the pipette tip. In some embodiments, the sample collection device is configured such that the collection tool, or the portion thereof, in the sample collection device is positioned to allow insertion of the pipette tip without interference from the collection tool, or the portion thereof. In some embodiments, the method further comprises inserting the pipette tip into the opening of the sample collection device. In some embodiments, inserting a pipette tip into the opening of the sample collection device does not contaminate a non-disposable portion of a pipette.

In some embodiments, provided is a method of obtaining at least a portion of a composition comprising a sample using a sample collection device, the method comprising: opening the sample collection device containing at least a portion of a collection tool and the composition comprising the sample; and drawing at least a portion of the composition comprising the sample out of the sample collection device via a pipette tip wherein the sample collection device comprises is described herein and comprises the inverted lip and the depression formed via the interior surface of the bottom portion of the elongated tube body of the sample collection device. In some embodiments, the collection tool, or the portion thereof, is not removed from the sample collection device prior to drawing at least a portion of the composition comprising the sample out of the sample collection device via the pipette tip. In some embodiments, the sample collection device is configured such that the collection tool, or the portion thereof, in the sample collection device is positioned to allow insertion of the pipette tip without interference from the collection tool, or the portion thereof. In some embodiments, the method further comprises inserting the pipette tip into the opening of the sample collection device. In some embodiments, inserting a pipette tip into the opening of the sample collection device does not contaminate a non-disposable portion of a pipette.

In some embodiments, the sample collection device is configured such that the collection tool, or the portion thereof, in the sample collection device is positioned to allow insertion of the pipette tip without interference from the collection tool, or the portion thereof. In some embodiments, the sample collection device is configured such that the collection tool, or the portion thereof, in the sample collection device is positioned to allow insertion of the pipette tip without contact to a sample collection portion on a distal portion of the collection tool. In some embodiments, the sample collection portion comprises a swab.

In some embodiments, opening the sample collection device comprises removing a cap of the sample collection device. In some embodiments, removing the cap is completed via a cap placement/removal device. In some embodiments, the cap placement/removal device is operated semi-automatically or automatically.

In some embodiments, opening the sample collection device comprises piercing a pierceable membrane of a cap. In some embodiments, the pierceable membrane can be pierced using a pipette tip, such as a disposible pipette tip. In some embodiments, the pierceable membrane can be pierced using a needle.

In some embodiments, the method further comprises reading a label on the sample collection device. In some embodiments, the label comprises information regarding a characteristic of the composition comprising the sample. In some embodiments, the characteristic is selected from the group consisting of volume, contents, source of the sample, and type of the sample. In some embodiments, reading the label on the sample collection device comprises scanning or capturing an image of the label. In some embodiments, the label comprises a barcode or QR code.

In some embodiments, the method further comprises subjecting at least a portion of the composition comprising the sample to a downstream analysis technique. In some embodiments, the downstream analysis technique comprises a purification protocol, such as a nucleic acid or polypeptide purification protocol. In some embodiments, the downstream analysis technique comprises an analyte detection protocol, such as PCR, nucleic acid sequencing, or mass spectrometry.

In some embodiments, the sample is a biological comprising a biological material. In some embodiments, the biological material is selected from the group consisting of a nucleic acid and a polypeptide. In some embodiments, the nucleic acid is an RNA or a DNA.

In some embodiments, the biological sample is of, or derived from, an individual. In some embodiments, the individual is a human.

In some embodiments, provided is a method comprising transporting a sample collection device described herein containing a fluid, wherein the sample collection device is configured using an inverted lip to substantially inhibit spillage of the fluid. In some embodiments, the sample collection device comprises an internal chamber defined by an elongated tube body and an opening in a top portion of the elongated tube body, and wherein the sample collection device is configured using an inverted lip to substantially inhibit spillage of a fluid into an area other than the internal chamber. In some embodiments, the fluid is a pre-loaded fluid, such as a reagent, that is loaded in the sample collection device prior to depositing a sample, such as a biological sample. In some embodiments, the pre-loaded fluid is loaded in the sample collection device by a first individual and a second individual collects the sample, such as a biological sample. In some embodiments, the inverted lip substantially inhibits spillage of a fluid when the sample collection device is capped. In some embodiments, an inverted lip that substantially inhibits spillage refers to a configuration that inhibits less than about 10%, such as less than about any of 9%, 8%, 7%, 6%, 5%, 4%, 3%, 2%, or 1%, loss of the fluid via the opening. In some embodiments, the sample collection device is transported to an individual for collecting a sample, such as a biological sample. In some embodiments, the sample collection device is transported to a processing facility.

In some embodiments, provided is a method of analyzing a sample, the method comprising obtaining the sample using (or from) a sample collection device described herein; and subjecting the sample to an analysis technique. In some embodiments, the sample is a biological sample comprising a biological material. In some embodiments, the biological material is selected from the group consisting of a nucleic acid and a polypeptide. In some embodiments, the nucleic acid is an RNA or a DNA. In some embodiments, the sample is of, or derived from, an individual. In some embodiments, the individual is a human. In some embodiments, the analysis technique comprises a purification protocol. In some embodiments, the analysis technique comprises an analyte detection protocol, such as using PCR, a nucleic acid sequencing technique, such as NGS, or mass spectrometry. In some embodiments, the analyte detection protocol is configured to detect a presence of an infectious agent or a disease. In some embodiments, the infection agent is a bacterium or a virus. In some embodiments, the virus is a corona virus, such as SARS-COV-2. In some embodiments, the presence of an infectious agent or a disease is detected via a biomarker.

### EXEMPLARY EMBODIMENTS

Embodiment 1. A sample collection device comprising an elongated tube body comprising an internal chamber, wherein the elongated tube body comprises a side wall portion, a top portion, and a bottom portion, wherein the elongated tube body has an interior surface and an exterior surface, wherein the top portion comprises an inverted lip forming an opening to the internal chamber, and wherein the inverted lip is configured to form a reservoir between at least a portion of the interior surface of the inverted lip and at least a portion of the interior surface of the side wall portion.
Embodiment 2. A sample collection device comprising an elongated tube body comprising an internal chamber, wherein the elongated tube body comprises a side wall portion, a top portion, and a bottom portion having an interior surface and an exterior surface, wherein the interior surface of the bottom portion comprises a slope forming a depression positioned away from a longitudinal central axis of the elongated tube body, and wherein the top portion comprises an opening.
Embodiment 3. A sample collection device comprising an elongated tube body comprising an internal chamber, wherein the elongated tube body comprises a side wall portion, an top portion, a bottom portion having an interior surface and an exterior surface, wherein the top portion comprises an inverted lip forming an opening to the internal chamber, wherein the inverted lip is configured to form a reservoir between at least a portion of the interior surface of the inverted lip and at least a portion of the interior surface of the side wall portion, and wherein the interior surface of the bottom portion comprises a slope forming a depression positioned away from a longitudinal central axis of the elongated tube body.
Embodiment 4. The sample collection device of any one of embodiments 1-3, wherein the volume of the reservoir is at least about 0.25 mL.
Embodiment 5. The sample collection device of any one of embodiments 1-4, wherein the volume of the reservoir is about 0.25 mL to about 5 mL.
Embodiment 6. The sample collection device of embodiment 4 or 5, wherein the volume of the reservoir is based on liquid capacity when the sample collection device is inverted relative to a longitudinal central axis of the elongated tube body.
Embodiment 7. The sample collection device of any one of embodiments 1-6, wherein the inverted lip and the elongated tube body are monolithic.
Embodiment 8. The sample collection device of any one of embodiments 1-6, wherein the inverted lip and the elongated tube body are separate pieces.
Embodiment 9. The sample collection device of embodiment 8, wherein the elongated tube body comprises a first material and the inverted lip comprises a second material.
Embodiment 10. The sample collection device of embodiment 9, wherein the first material of the elongated tube body and the second material of the inverted lip are different.
Embodiment 11. The sample collection device of embodiment 9, wherein the first material of the elongated tube body and the second material of the inverted lip are the same.
Embodiment 12. The sample collection device of any one of embodiments 9-11, wherein the first material and the second material comprise a thermoformable polymer.
Embodiment 13. The sample collection device of any one of embodiments 8-12, wherein the side wall portion comprises a feature configured to interface with the inverted lip, wherein the interface between the side wall portion and the inverted lip forms a fluid-tight seal.
Embodiment 14. The sample collection device of any one of embodiments 1-13, wherein at least a portion of the exterior surface of the inverted lip comprises a taper in the direction of the top portion to the bottom portion.
Embodiment 15. The sample collection device of any one of embodiments 1-14, wherein at least a portion of the exterior surface of the inverted lip is configured to interface with a disposable pipette tip.
Embodiment 16. The sample collection device of any one of embodiments 1-15, wherein the exterior surface of the inverted lip is a substantially smooth surface.
Embodiment 17. The sample collection device of any one of embodiments 1-15, wherein the exterior surface of the inverted lip comprises at least one rib extending in the direction of a longitudinal central axis of the elongated tube body.
Embodiment 18. The sample collection device of any one of embodiments 1-16, wherein the opening of the top portion has a largest cross-sectional dimension that is equal to or less than the largest cross-sectional dimension of the internal surface of the side wall portion, wherein the cross-sectional dimension of the opening is substantially perpendicular to a longitudinal central axis of the elongated tube body.
Embodiment 19. The sample collection device of any one of embodiments 1-17, wherein the opening of the top portion has a largest cross-sectional dimension of about 3 mm to about 25 mm.
Embodiment 20. The sample collection device of any one of embodiments 1-19, wherein the opening of the top portion has a largest cross-sectional dimension of about 18 mm or less.
Embodiment 21. The sample collection device of any one of embodiments 1-20, wherein the ratio between the opening of the top portion and the largest cross-sectional dimension of the internal surface of the side wall portion is about 8:10 to about 1:10.
Embodiment 22. The sample collection device of any one of embodiments 1-21, wherein the ratio between the height of the elongated tube body and the largest cross-sectional dimension, as measured perpendicular to a longitudinal central axis of the elongated tube body, of the internal surface of the side wall portion is at least about 3:1 to about 20:1.
Embodiment 23. The sample collection device of any one of embodiments 1-22, wherein the ratio between the opening of the top portion and the height of the inverted lip is at least about 1:10 to about 10:1.
Embodiment 24. The sample collection device of any one of embodiments 1-23, wherein the ratio between the opening of the elongated tube body formed by the inverted lip and the largest cross-sectional dimensional, as measured perpendicular to a longitudinal central axis of the elongated tube body, of the internal surface of the sidewall portion is about 8:10 to about 1:10.
Embodiment 25. The sample collection device of any one of embodiments 1-24, wherein the top portion and the bottom portion are configured such that a disposable pipette tip will not contact the interior surface of the bottom portion when the disposable pipette tip is inserted in the opening of the top portion.
Embodiment 26. The sample collection device of any one of embodiments 1-25, wherein at least a portion of the exterior surface of the side wall portion is cylindrical.
Embodiment 27. The sample collection device of any one of embodiments 1-24, wherein the length of the elongated tube body is about 2 cm to about 10 cm, as measured parallel to a longitudinal central axis of the elongated tube body.
Embodiment 28. The sample collection device of any one of embodiments 1-27, wherein a cross-section of the exterior surface of the side wall portion is circular.
Embodiment 29. The sample collection device of any one of embodiments 1-28, wherein at least a portion of the exterior surface of the bottom portion of the elongated tube body forms a substantially flat bottom relative to a plane perpendicular to a longitudinal central axis of the elongated tube body.
Embodiment 30. The sample collection device of any one of embodiments 1-29, wherein the internal chamber has a volume of at least about 1.5 mL.
Embodiment 31. The sample collection device of any one of embodiments 1-30, further comprising a cap.
Embodiment 32. The sample collection device of embodiment 31, wherein the cap is a threaded cap.
Embodiment 33. The sample collection device of embodiment 32, wherein the exterior surface of the elongated tube body comprises a thread configured to interface with the threaded cap.
Embodiment 34. The sample collection device of embodiment 31, wherein the cap is a push cap.
Embodiment 35. The sample collection device of embodiment 34, wherein the exterior surface and/or interior surface of the elongated tube body comprises a feature configured to interface with the push cap.
Embodiment 36. The sample collection device of any one of embodiments 31-35, wherein the cap is configured to interface with at least a portion of the inverted lip.
Embodiment 37. The sample collection device of any one of embodiments 31-36, wherein the cap comprises a feature configured to interface with a cap placement/removal device.
Embodiment 38. The sample collection device of any one of embodiments 31-37, wherein the cap comprises a pierceable membrane.
Embodiment 39. A kit comprising a sample collection device of any one of embodiments 1-38, and a collection tool.
Embodiment 40. The kit of embodiment 39, wherein the collection tool comprises a sample collection portion, a stem portion, and a handle portion, wherein the sample collection portion is on a distal portion of the collection tool, wherein the handle portion is on proximal portion of the collection tool, and wherein the stem portion connects the sample collection portion and the handle portion.
Embodiment 41. The kit of embodiment 40, wherein the collection tool comprises a break point feature configured to break the stem portion at a pre-determined location to separate the sample collection portion and the handle portion.
Embodiment 42. The kit of any one of embodiments 39-41, wherein the collection tool is a collection swab.
Embodiment 43. The kit of any one of embodiments 39-42, wherein the size of the collection tool, or portion thereof, configured to be deposited in the sample collection device is based on the size of an internal chamber of the sample collection device.
Embodiment 44. The kit of any one of embodiments 39-43, wherein the size of the collection tool, or portion thereof, configured to be deposited in the sample collection device is based on the size of an internal chamber of the sample collection device and the position of an inverted lip relative to a bottom portion of an elongated tube body of the sample collection device, such that the collection tool, or portion thereof, configured to be deposited in the sample collection device sits below the inverted lip when in contact with the bottom portion of the elongated tube body.
Embodiment 45. The kit of any one of embodiments 39-44, further comprising instructions for collecting a sample.
Embodiment 46. A method of collecting a sample, the method comprising: obtaining the sample using a collection tool; and inserting at least a portion of the collection tool in a sample collection device via an opening, wherein the sample collection device is selected from any one of embodiments 1 and 3-38, and wherein the sample collection device is configured using the inverted lip to substantially inhibit spillage of a fluid when the opening of the sample collection device is not sealed by a cap.
Embodiment 47. A method of collecting a sample, the method comprising: obtaining the sample using a collection tool; and inserting at least a portion of the collection tool in a sample collection device via an opening, wherein the sample collection device is selected from any one of embodiments 2-38, wherein the sample collection device is configured to accept at least a portion of the collection tool in the depression formed by the interior surface of the bottom portion of the elongated tube body of the sample collection device, and wherein the depression is positioned away from a longitudinal central axis of the elongated tube body.
Embodiment 48. A method of collecting a sample, the method comprising: obtaining the sample using a collection tool; and inserting at least a portion of the collection tool in a sample collection device via an opening, wherein the sample collection device is selected from any one of embodiments 3-38, wherein the sample collection device is configured using the inverted lip to substantially inhibit spillage of a fluid when the opening of the sample collection device is not sealed by a cap, wherein the sample collection device is configured to accept at least a portion of the collection tool in the depression formed by the interior surface of the bottom portion of the elongated tube body of the sample collection device, and wherein the depression is positioned away from a longitudinal central axis of the elongated tube body.
Embodiment 49. The method of any one of embodiments 46-48, further comprising closing the sample collection device using the cap.
Embodiment 50. The method of embodiment 49, wherein the cap comprises a pierceable membrane.
Embodiment 51. The method of any one of embodiments 46-49, further comprising depositing at least a portion of the collection tool in the sample collection device.
Embodiment 52. The method of embodiment 51, wherein the collection tool, or portion thereof, is configured such that when deposited in the sample collection device the collection tool, or portion thereof, is contained in the internal chamber below the opening and proximal to the bottom portion of the elongated tube body of the sample collection device.
Embodiment 53. The method of embodiment 51 or 50, wherein depositing at least a portion of the collection tool comprises exerting a force on the collection tool to break off the portion thereof via a break point feature.
Embodiment 54. The method of any one of embodiments 46-53, wherein the collection tool is a collection swab.
Embodiment 55. The method of any one of embodiments 46-54, wherein the sample is a biological sample comprising a biological material.
Embodiment 56. The method of embodiment 55, wherein the biological material is selected from the group consisting of a nucleic acid and a polypeptide.
Embodiment 57. The method of embodiment 56, wherein the nucleic acid is an RNA or a DNA.
Embodiment 58. The method of any one of embodiments 46-57, wherein the sample is of, or derived from, an individual.
Embodiment 59. The method of embodiment 58, wherein the individual is a human.
Embodiment 60. The method of embodiment 58 or 59, wherein obtaining the sample comprises sampling the individual, or a substance derived therefrom, using the collection tool.
Embodiment 61. The method of any one of embodiments 46-60, further comprising obtaining the sample collection device and collection tool from a point of origination.
Embodiment 62. The method of any one of embodiments 46-61, further comprising shipping the capped sample collection device to a destination.
Embodiment 63. The method of embodiment 62, wherein the destination and the point of origination are the same location.
Embodiment 64. A method of obtaining at least a portion of a composition comprising a sample contained in a sample collection device, the method comprising: opening the sample collection device containing the composition comprising the sample; and drawing at least a portion of the composition comprising the sample out of the sample collection device via a pipette tip, wherein the sample collection device is selected from any one of embodiments 1 and 3-38 and comprises the inverted lip.
Embodiment 65. A method of obtaining at least a portion of a composition comprising a sample contained in a sample collection device, the method comprising: opening the sample collection device containing at least a portion of a collection tool and the composition comprising the sample; and drawing at least a portion of the composition comprising the sample out of the sample collection device via a pipette tip, wherein the sample collection device is selected from any one of embodiments 2-38 and comprises the depression formed via the interior surface of the bottom portion of the elongated tube body of the sample collection device.
Embodiment 66. A method of obtaining at least a portion of a composition comprising a sample using a sample collection device, the method comprising: opening the sample collection device containing at least a portion of a collection tool and the composition comprising the sample; and drawing at least a portion of the composition comprising the sample out of the sample collection device via a pipette tip, wherein the sample collection device comprises is selected from any one of embodiments 3-38 and comprises the inverted lip and the depression formed via the interior surface of the bottom portion of the elongated tube body of the sample collection device.
Embodiment 67. The method of embodiment 65 or 66, wherein the collection tool, or the portion thereof, is not removed from the sample collection device prior to drawing at least a portion of the composition comprising the sample out of the sample collection device via the pipette tip.
Embodiment 68. The method of embodiment 67, wherein the sample collection device is configured such that the collection tool, or the portion thereof, in the sample collection device is positioned to allow insertion of the pipette tip without interference from the collection tool, or the portion thereof.
Embodiment 69. The method of any one of embodiments 64-68, further comprising inserting the pipette tip into the opening of the sample collection device.
Embodiment 70. The method of any one of embodiments 64-69, wherein the composition comprises substantially the same amount of a pre-loaded fluid as in the sample collection device prior to addition of the sample.
Embodiment 71. The method of any one of embodiments 64-70, wherein opening the sample collection device comprises removing a cap of the sample collection device.
Embodiment 72. The method of embodiment 71, wherein removing the cap is completed via a cap placement/removal device.
Embodiment 73. The method of any one of embodiments 64-70, wherein opening the sample collection device comprises piercing a pierceable membrane of a cap.
Embodiment 74. The method of any one of embodiments 64-73, further comprising reading a label on the sample collection device.
Embodiment 75. The method of embodiment 74, wherein the label comprises information regarding a characteristic of the composition comprising the sample.
Embodiment 76. The method of embodiment 75, wherein the characteristic is selected from the group consisting of volume, contents, source of the sample, and type of the sample.
Embodiment 77. The method of any one of embodiments 64-76, further comprising subjecting at least a portion of the composition comprising the sample to a downstream analysis technique.
Embodiment 78. The method of embodiment 77, wherein the downstream analysis technique comprises a purification protocol.
Embodiment 79. The method of embodiment 77 or 78, wherein the downstream analysis technique comprises an analyte detection protocol.
Embodiment 80. The method of any one of embodiments 64-79, wherein the sample is a biological sample comprising a biological material.
Embodiment 81. The method of embodiment 80, wherein the biological material is selected from the group consisting of a nucleic acid and a polypeptide.
Embodiment 82. The method of embodiment 81, wherein the nucleic acid is an RNA or a DNA.
Embodiment 83. The method of any one of embodiments 64-82, wherein the sample is of, or derived from, an individual.
Embodiment 84. The method of embodiment 83, wherein the individual is a human.

What is claimed is:

1. A sample collection device comprising an elongated tube body comprising an internal chamber,
wherein the elongated tube body comprises a side wall portion, a top portion, and a bottom portion,
wherein the elongated tube body has an interior surface and an exterior surface,
wherein the top portion comprises an inverted lip forming an opening to the internal chamber, and
wherein the inverted lip is configured to form a reservoir between at least a portion of the interior surface of the inverted lip and at least a portion of the interior surface of the side wall portion.

2. A sample collection device comprising an elongated tube body comprising an internal chamber,
wherein the elongated tube body comprises a side wall portion, a top portion, and a bottom portion having an interior surface and an exterior surface,
wherein the interior surface of the bottom portion comprises a slope forming a depression positioned away from a longitudinal central axis of the elongated tube body, and
wherein the top portion comprises an opening.

3. A sample collection device comprising an elongated tube body comprising an internal chamber,
wherein the elongated tube body comprises a side wall portion, an top portion, a bottom portion having an interior surface and an exterior surface,
wherein the top portion comprises an inverted lip forming an opening to the internal chamber,
wherein the inverted lip is configured to form a reservoir between at least a portion of the interior surface of the inverted lip and at least a portion of the interior surface of the side wall portion, and
wherein the interior surface of the bottom portion comprises a slope forming a depression positioned away from a longitudinal central axis of the elongated tube body.

4. The sample collection device of any one of claims 1-3, wherein the volume of the reservoir is at least about 0.25 mL.

5. The sample collection device of any one of claims 1-4, wherein the volume of the reservoir is about 0.25 mL to about 5 mL.

6. The sample collection device of claim 4 or 5, wherein the volume of the reservoir is based on liquid capacity when the sample collection device is inverted relative to a longitudinal central axis of the elongated tube body.

7. The sample collection device of any one of claims 1-6, wherein the inverted lip and the elongated tube body are monolithic.

8. The sample collection device of any one of claims 1-6, wherein the inverted lip and the elongated tube body are separate pieces.

9. The sample collection device of claim 8, wherein the elongated tube body comprises a first material and the inverted lip comprises a second material.

10. The sample collection device of claim 9, wherein the first material of the elongated tube body and the second material of the inverted lip are different.

11. The sample collection device of claim 9, wherein the first material of the elongated tube body and the second material of the inverted lip are the same.

12. The sample collection device of any one of claims 9-11, wherein the first material and the second material comprise a thermoformable polymer.

13. The sample collection device of any one of claims 8-12, wherein the side wall portion comprises a feature configured to interface with the inverted lip, wherein the interface between the side wall portion and the inverted lip forms a fluid-tight seal.

14. The sample collection device of any one of claims 1-13, wherein at least a portion of the exterior surface of the inverted lip comprises a taper in the direction of the top portion to the bottom portion.

15. The sample collection device of any one of claims 1-14, wherein at least a portion of the exterior surface of the inverted lip is configured to interface with a disposable pipette tip.

16. The sample collection device of any one of claims 1-15, wherein the exterior surface of the inverted lip is a substantially smooth surface.

17. The sample collection device of any one of claims 1-15, wherein the exterior surface of the inverted lip comprises at least one rib extending in the direction of a longitudinal central axis of the elongated tube body.

18. The sample collection device of any one of claims 1-16, wherein the opening of the top portion has a largest cross-sectional dimension that is equal to or less than the largest cross-sectional dimension of the internal surface of the side wall portion, wherein the cross-sectional dimension of the opening is substantially perpendicular to a longitudinal central axis of the elongated tube body.

19. The sample collection device of any one of claims 1-17, wherein the opening of the top portion has a largest cross-sectional dimension of about 3 mm to about 25 mm.

20. The sample collection device of any one of claims 1-19, wherein the opening of the top portion has a largest cross-sectional dimension of about 18 mm or less.

21. The sample collection device of any one of claims 1-20, wherein the ratio between the opening of the top portion and the largest cross-sectional dimension of the internal surface of the side wall portion is about 8:10 to about 1:10.

22. The sample collection device of any one of claims 1-21, wherein the ratio between the height of the elongated tube body and the largest cross-sectional dimension, as measured perpendicular to a longitudinal central axis of the elongated tube body, of the internal surface of the side wall portion is at least about 3:1 to about 20:1.

23. The sample collection device of any one of claims 1-22, wherein the ratio between the opening of the top portion and the height of the inverted lip is at least about 1:10 to about 10:1.

24. The sample collection device of any one of claims 1-23, wherein the ratio between the opening of the elongated tube body formed by the inverted lip and the largest cross-sectional dimensional, as measured perpendicular to a longitudinal central axis of the elongated tube body, of the internal surface of the sidewall portion is about 8:10 to about 1:10.

25. The sample collection device of any one of claims 1-24, wherein the top portion and the bottom portion are configured such that a disposable pipette tip will not contact the interior surface of the bottom portion when the disposable pipette tip is inserted in the opening of the top portion.

26. The sample collection device of any one of claims 1-25, wherein at least a portion of the exterior surface of the side wall portion is cylindrical.

27. The sample collection device of any one of claims 1-24, wherein the length of the elongated tube body is about 2 cm to about 10 cm, as measured parallel to a longitudinal central axis of the elongated tube body.

28. The sample collection device of any one of claims 1-27, wherein a cross-section of the exterior surface of the side wall portion is circular.

29. The sample collection device of any one of claims 1-28, wherein at least a portion of the exterior surface of the bottom portion of the elongated tube body forms a substantially flat bottom relative to a plane perpendicular to a longitudinal central axis of the elongated tube body.

30. The sample collection device of any one of claims 1-29, wherein the internal chamber has a volume of at least about 1.5 mL.

31. The sample collection device of any one of claims 1-30, further comprising a cap.

32. The sample collection device of claim 31, wherein the cap is a threaded cap.

33. The sample collection device of claim 32, wherein the exterior surface of the elongated tube body comprises a thread configured to interface with the threaded cap.

34. The sample collection device of claim 31, wherein the cap is a push cap.

35. The sample collection device of claim 34, wherein the exterior surface and/or interior surface of the elongated tube body comprises a feature configured to interface with the push cap.

36. The sample collection device of any one of claims 31-35, wherein the cap is configured to interface with at least a portion of the inverted lip.

37. The sample collection device of any one of claims 31-36, wherein the cap comprises a feature configured to interface with a cap placement/removal device.

38. The sample collection device of any one of claims 31-37, wherein the cap comprises a pierceable membrane.

39. A kit comprising a sample collection device of any one of claims 1-38, and a collection tool.

40. The kit of claim 39, wherein the collection tool comprises a sample collection portion, a stem portion, and a handle portion, wherein the sample collection portion is on a distal portion of the collection tool, wherein the handle portion is on proximal portion of the collection tool, and wherein the stem portion connects the sample collection portion and the handle portion.

41. The kit of claim 40, wherein the collection tool comprises a break point feature configured to break the stem portion at a pre-determined location to separate the sample collection portion and the handle portion.

42. The kit of any one of claims 39-41, wherein the collection tool is a collection swab.

43. The kit of any one of claims 39-42, wherein the size of the collection tool, or portion thereof, configured to be deposited in the sample collection device is based on the size of an internal chamber of the sample collection device.

44. The kit of any one of claims 39-43, wherein the size of the collection tool, or portion thereof, configured to be deposited in the sample collection device is based on the size of an internal chamber of the sample collection device and the position of an inverted lip relative to a bottom portion of an elongated tube body of the sample collection device, such that the collection tool, or portion thereof, configured to be deposited in the sample collection device sits below the inverted lip when in contact with the bottom portion of the elongated tube body.

45. The kit of any one of claims 39-44, further comprising instructions for collecting a sample.

46. A method of collecting a sample, the method comprising:
obtaining the sample using a collection tool; and
inserting at least a portion of the collection tool in a sample collection device via an opening,
wherein the sample collection device is selected from any one of claims 1 and 3-38, and
wherein the sample collection device is configured using the inverted lip to substantially inhibit spillage of a fluid when the opening of the sample collection device is not sealed by a cap.

47. A method of collecting a sample, the method comprising:
obtaining the sample using a collection tool; and
inserting at least a portion of the collection tool in a sample collection device via an opening,
wherein the sample collection device is selected from any one of claims 2-38,
wherein the sample collection device is configured to accept at least a portion of the collection tool in the depression formed by the interior surface of the bottom portion of the elongated tube body of the sample collection device, and
wherein the depression is positioned away from a longitudinal central axis of the elongated tube body.

48. A method of collecting a sample, the method comprising:
obtaining the sample using a collection tool; and
inserting at least a portion of the collection tool in a sample collection device via an opening,
wherein the sample collection device is selected from any one of claims 3-38,
wherein the sample collection device is configured using the inverted lip to substantially inhibit spillage of a fluid when the opening of the sample collection device is not sealed by a cap,
wherein the sample collection device is configured to accept at least a portion of the collection tool in the depression formed by the interior surface of the bottom portion of the elongated tube body of the sample collection device, and
wherein the depression is positioned away from a longitudinal central axis of the elongated tube body.

49. The method of any one of claims 46-48, further comprising closing the sample collection device using the cap.

50. The method of claim 49, wherein the cap comprises a pierceable membrane.

51. The method of any one of claims 46-49, further comprising depositing at least a portion of the collection tool in the sample collection device.

52. The method of claim 51, wherein the collection tool, or portion thereof, is configured such that when deposited in the sample collection device the collection tool, or portion thereof, is contained in the internal chamber below the opening and proximal to the bottom portion of the elongated tube body of the sample collection device.

53. The method of claim 51 or 50, wherein depositing at least a portion of the collection tool comprises exerting a force on the collection tool to break off the portion thereof via a break point feature.

54. The method of any one of claims 46-53, wherein the collection tool is a collection swab.

55. The method of any one of claims 46-54, wherein the sample is a biological sample comprising a biological material.

56. The method of claim 55, wherein the biological material is selected from the group consisting of a nucleic acid and a polypeptide.

57. The method of claim 56, wherein the nucleic acid is an RNA or a DNA.

58. The method of any one of claims 46-57, wherein the sample is of, or derived from, an individual.

59. The method of claim 58, wherein the individual is a human.

60. The method of claim 58 or 59, wherein obtaining the sample comprises sampling the individual, or a substance derived therefrom, using the collection tool.

61. The method of any one of claims 46-60, further comprising obtaining the sample collection device and collection tool from a point of origination.

62. The method of any one of claims 46-61, further comprising shipping the capped sample collection device to a destination.

63. The method of claim 62, wherein the destination and the point of origination are the same location.

64. A method of obtaining at least a portion of a composition comprising a sample contained in a sample collection device, the method comprising:
opening the sample collection device containing the composition comprising the sample; and
drawing at least a portion of the composition comprising the sample out of the sample collection device via a pipette tip,
wherein the sample collection device is selected from any one of claims 1 and 3-38 and comprises the inverted lip.

65. A method of obtaining at least a portion of a composition comprising a sample contained in a sample collection device, the method comprising:
opening the sample collection device containing at least a portion of a collection tool and the composition comprising the sample; and
drawing at least a portion of the composition comprising the sample out of the sample collection device via a pipette tip,
wherein the sample collection device is selected from any one of claims 2-38 and comprises the depression formed via the interior surface of the bottom portion of the elongated tube body of the sample collection device.

66. A method of obtaining at least a portion of a composition comprising a sample using a sample collection device, the method comprising:
opening the sample collection device containing at least a portion of a collection tool and the composition comprising the sample; and
drawing at least a portion of the composition comprising the sample out of the sample collection device via a pipette tip,
wherein the sample collection device comprises is selected from any one of claims 3-38 and comprises the inverted lip and the depression formed via the interior surface of the bottom portion of the elongated tube body of the sample collection device.

67. The method of claim 65 or 66, wherein the collection tool, or the portion thereof, is not removed from the sample collection device prior to drawing at least a portion of the composition comprising the sample out of the sample collection device via the pipette tip.

68. The method of claim 67, wherein the sample collection device is configured such that the collection tool, or the portion thereof, in the sample collection device is positioned to allow insertion of the pipette tip without interference from the collection tool, or the portion thereof.

69. The method of any one of claims 64-68, further comprising inserting the pipette tip into the opening of the sample collection device.

70. The method of any one of claims 64-69, wherein the composition comprises substantially the same amount of a pre-loaded fluid as in the sample collection device prior to addition of the sample.

71. The method of any one of claims 64-70, wherein opening the sample collection device comprises removing a cap of the sample collection device.

72. The method of claim 71, wherein removing the cap is completed via a cap placement/removal device.

73. The method of any one of claims 64-70, wherein opening the sample collection device comprises piercing a pierceable membrane of a cap.

74. The method of any one of claims 64-73, further comprising reading a label on the sample collection device.

75. The method of claim 74, wherein the label comprises information regarding a characteristic of the composition comprising the sample.

76. The method of claim 75, wherein the characteristic is selected from the group consisting of volume, contents, source of the sample, and type of the sample.

77. The method of any one of claims 64-76, further comprising subjecting at least a portion of the composition comprising the sample to a downstream analysis technique.

78. The method of claim 77, wherein the downstream analysis technique comprises a purification protocol.

79. The method of claim 77 or 78, wherein the downstream analysis technique comprises an analyte detection protocol.

80. The method of any one of claims 64-79, wherein the sample is a biological sample comprising a biological material.

81. The method of claim 80, wherein the biological material is selected from the group consisting of a nucleic acid and a polypeptide.

82. The method of claim 81, wherein the nucleic acid is an RNA or a DNA.

83. The method of any one of claims 64-82, wherein the sample is of, or derived from, an individual.

84. The method of claim 83, wherein the individual is a human.
